(19) United States
(12) Patent Application Publication
(10) Pub. No.: US 2024/0246812 A1
(43) Pub. Date: Jul. 25, 2024

(54) ULTRATHIN FREE-STANDING SOLID STATE MEMBRANE CHIPS AND METHODS OF MAKING

(71) Applicant: UNIVERSITY OF WASHINGTON, Seattle, WA (US)

(72) Inventors: Bo Zhang, Seattle, WA (US); Todd Anderson, Seattle, WA (US); Chris McAllister, Seattle, WA (US)

(73) Assignee: UNIVERSITY OF WASHINGTON, Seattle, WA (US)

(21) Appl. No.: 18/564,996

(22) PCT Filed: Jun. 22, 2022
(86) PCT No.: PCT/US2022/034558

§ 371 Date: Nov. 28, 2023

Related U.S. Application Data
(60) Provisional application No. 63/213,621, filed on Jun. 22, 2021.

(51) Int. Cl. B81C 1/00; B81B 7/00; C03C 15/00; C03C 17/22; C03C 17/245; C23C 16/02; C23C 16/34; C23C 16/40; C23C 16/50; C23C 16/56; G01N 33/487

(52) U.S. Cl. CPC B81C 1/00341; B81B 7/00; C03C 15/00; C03C 17/225; C03C 17/245; C23C 16/0227; C23C 16/345; C23C 16/401; C23C 16/50; C23C 16/56; G01N 33/48721; B81B 2203/0127; B81C 2201/0132; B81C 2201/0133; B81C 2201/0176; B81C 2201/019; C03C 2218/152; C03C 2218/153; C03C 2218/32

(57) ABSTRACT

An ultrathin free-standing solid state membrane, including an etched well on a glass wafer, and a layer of SiX deposited on a backside of the etched well on the glass wafer.

### CROSS-REFERENCE TO RELATED APPLICATION

This application claims the benefit of U.S. Provisional Application No. 63/213,621 filed Jun. 22, 2021, the entire contents of which are hereby expressly incorporated by reference.

### BACKGROUND

Solid state nanopores on glass chips for electronic DNA sequencing, single-nanoparticle analysis, and other ordered analysis applications have gained popularity due to a host of advantages versus other nanopores, such as biological nanopores. These advantages include, tunable pore size and shape with sub nanometer resolution, mechanical robustness, superior thermal and chemical stability over a wide range of conditions (i.e., pH, temperature concentration), parallel fabrication techniques that can easily produce many identical setups, and integration compatibility with sophisticated electronics and optical readout systems.

Though solid state nanopores offer advantages, there is still more to offer regarding precision, reliability, etc. through advances in fabrication of free-standing solid-state membranes into which they are created via various means and methods. Thus, free-standing solid-state membranes, and methods of making free-standing solid state membranes having more precision and reliability are needed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, an ultrathin free-standing solid state membrane, including an etched well on a glass wafer, and a layer of SiX deposited on a backside of the etched well on the glass wafer is disclosed.

In another aspect, a method of making an ultrathin free-standing solid state membrane, the method comprising bonding silicon with a first side of a glass wafer, depositing a gold layer on a second side of the glass wafer, patterning the gold layer, etching the glass wafer to form a well, depositing a layer of SiX onto the second side of the glass wafer, and removing the silicon is disclosed.

In yet another aspect, a method of using the membrane of claim 1 for MEMS device scaffolding, DNA sequencing, TEM imaging, microparticle analysis, nanoparticle analysis, medicinal applications, environmental applications, electrochemical applications, or mechanical applications is disclosed.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example ultrathin free-standing solid-state membrane, in accordance with the present technology;

FIG. 1B is a side view of another example ultrathin free-standing solid-state membrane, in accordance with the present technology;

FIGS. 2A-2G illustrate an example method of making an ultrathin free-standing solid-state membrane, in accordance with the present technology;

FIG. 3A is an example glass wafer having dicing markers, in accordance with the present technology;

FIG. 3B is a plurality of glass chips formed from the glass wafer along the dicing markers of FIG. 3A, in accordance with the present technology;

FIG. 4 is an SEM image of an example well of an ultrathin free-standing solid-state membrane, in accordance with the present technology;

FIG. 5 is a composite of four brightfield micrographs of four different wells located on a single glass wafer, in accordance with the present technology;

FIG. 6 is an SEM image of the thickness of an example ultrathin free-standing solid-state membrane, in accordance with the present technology;

FIG. 7A is a brightfield micrograph of a silicon oxide ultrathin free-standing solid-state membrane, in accordance with the present technology;

FIG. 7B is a brightfield micrograph of another silicon oxide ultrathin free-standing solid-state membrane, in accordance with the present technology; and

FIG. 8A-8D are SEM images of example hydrofluoric acid (HF) etched glass chips, in accordance with the present technology.

### DETAILED DESCRIPTION

Described herein are ultrathin free standing solid-state membranes (or membranes) and methods for producing glass wafers or chips including one or more ultrathin solid-state membranes. In some embodiments, the ultrathin freestanding solid-state membranes are formed from silicon nitride or silicon oxide. In some embodiments, the membrane has a variety of uses, including electronic-based DNA sequencing and single nanoparticle analysis, among many other applications. In some embodiments, the membranes are formed on borosilicate glass, quartz glass, or a combination thereof. The use of borosilicate or quartz glass enables low-noise electric recordings, in comparison with silicon-based chips. In some embodiments, the membranes are formed on one or more glass wafers. In some embodiments, the one or more glass wafers are diced into one or more glass chips. Hundreds or more glass chips including a membrane can be created from a single glass wafer. In some embodiments, the silicon-nitrogen membrane or silicon-oxygen membranes range from tens to hundreds of nanometers in thickness, as described in detail herein.

In one aspect, an ultrathin free-standing solid state membrane, including an etched well on a glass wafer, and a layer of SiX deposited on a backside of the etched well on the glass wafer is disclosed.

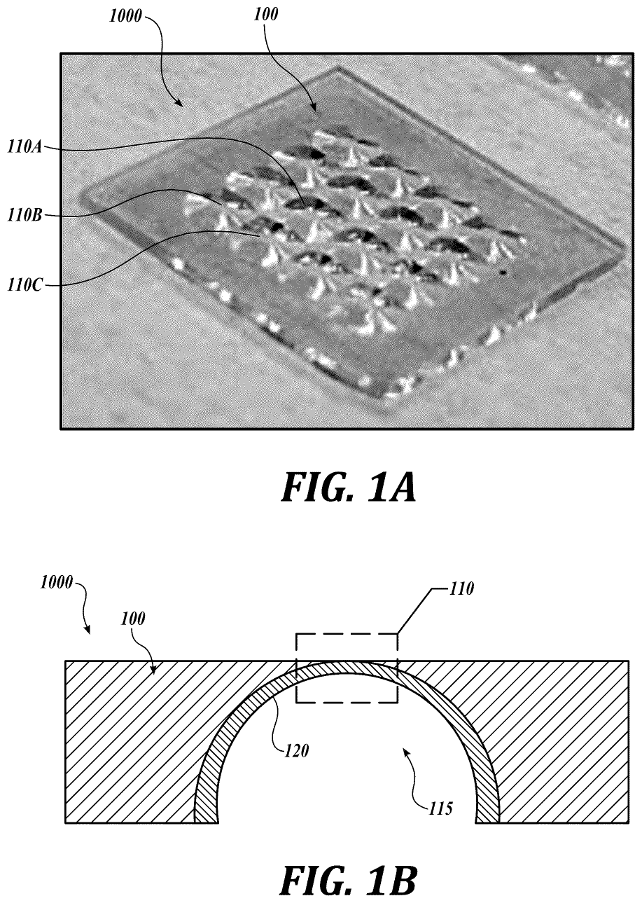

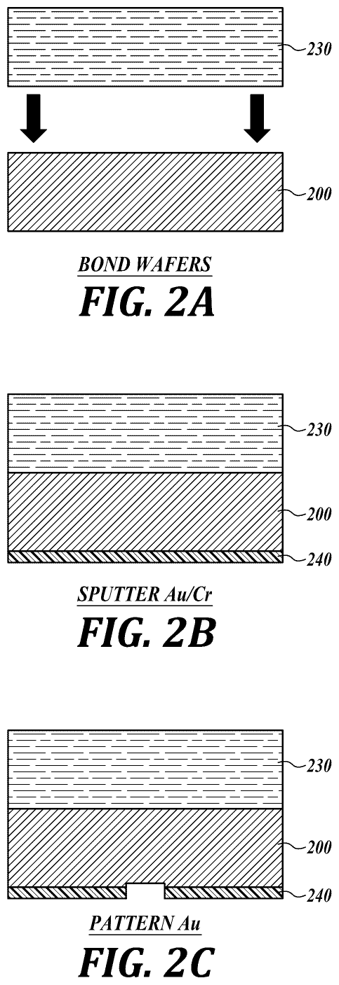

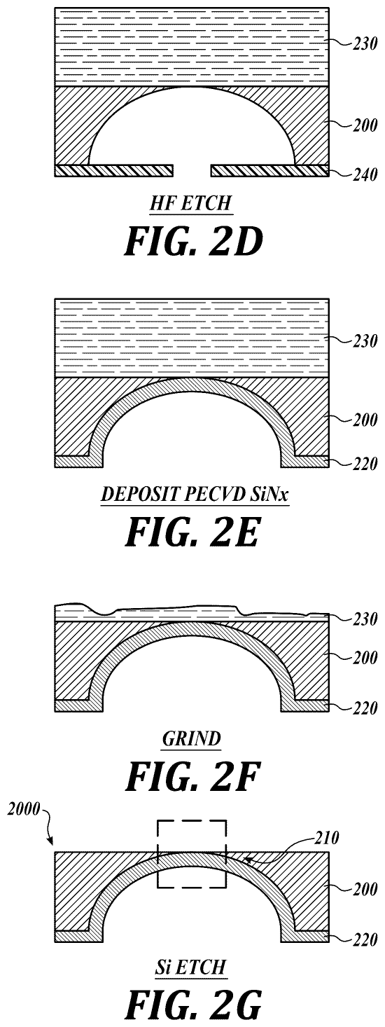

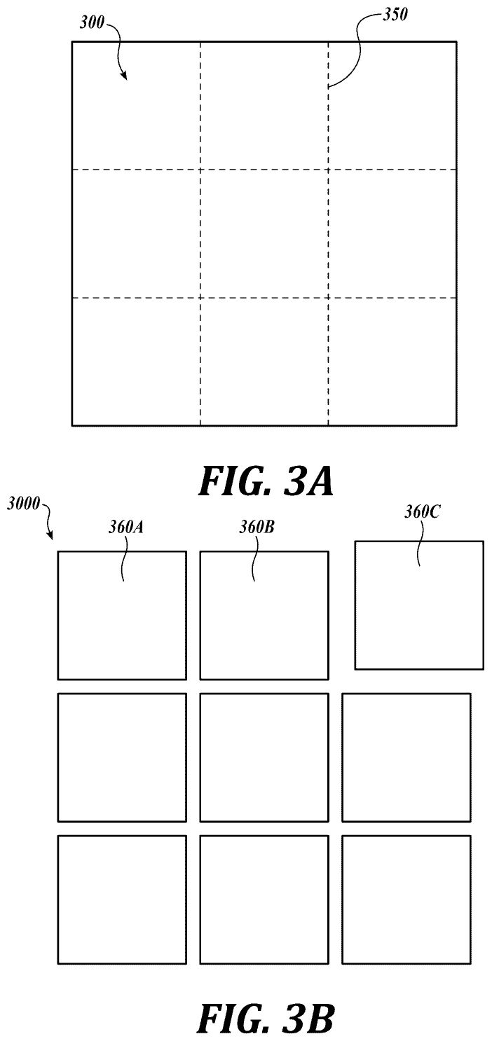

FIG. 1A is an example ultrathin free-standing solid-state membrane 1000, in accordance with the present technology. In some embodiments, the membrane 1000 includes a glass wafer 100 and a plurality of wells 110A, 110B, 110C. In some embodiments, the glass wafer 100 is a glass chip that is part of a larger glass wafer (as shown in FIGS. 2A-2G). In some embodiments, the glass chip 100 is diced from a larger glass wafer as shown in FIGS. 3A-3B.

In some embodiments, the plurality of wells 110A, 110B, 110C form an array on the glass wafer 100. In some embodiments, a layer of SiX is deposited on the backside of the glass wafer 100 (as shown and described in FIG. 1B). SiX, as defined herein, is a silicon compound, such as a silicon-oxygen compound or a silicon-nitrogen compound. In some embodiments, the SiX is a silicon-nitrogen compound, such as silicon nitride. In some embodiments, the Six is a silicon-oxygen compound, such as silicon oxide. The plurality of wells 110A, 110B, 110C may expose the Six on the backside of the glass chip 100. In some embodiments, the etched wells 110A, 110B, 110C include a plurality of nanopores. In some embodiments, each etched well 110 of the plurality of etched wells 110A, 110B, 110C includes a plurality of nanopores. In some embodiments, the etched wells 110A, 110B, 110C range from about 1 μm to 5000 μm in diameter at the front side of the glass wafer 100. As used herein, the term “about” should be construed as +/−5% of the disclosed amount.

In operation, the SiX is chemically robust, has a high thermal stability, acts as an insulator, and is carbon free. Additionally, the membrane 1000 is electron transparent for low background TEM imaging, has a high mechanical stability, and is machinable. Because of this, the membrane 1000 may be configured to be used as a scaffold to build MEMs devices, in addition to numerous other applications. Further, the membrane 1000 may be configured to be used in medicinal, environmental, and mechanical applications due to its ability to separate two regions of space or fluid. The nanoscale wells 110A, 110B, 110C on the glass chip 100 can control molecular flow through the membrane 1000 which, when monitored, enables detection of small species passing through the pores. While conventional membranes made of silicon can be problematic in electrochemical biosensing and detection due to relatively high capacitance (and thus high noise), the glass-based membrane 1000 has a significantly lower capacitance. Because of this, ionic flow detection experiments, along with other electrochemical biosensing detection experiments can be performed with the membrane 1000.

FIG. 1B is a side view of another example ultrathin free-standing solid-state membrane 1000, in accordance with the present technology. In some embodiments, the membrane 1000 includes a glass chip 100, including a well 110, a layer of SiX 120, and a cavity 115. In some embodiments, the glass chip 100 has a front side and a back side, illustrated as the top plane and the bottom plane of the glass chip 100, respectively. In some embodiments, SiX 120 is coated on the back side of the glass chip 100, as illustrated in FIG. 1B.

In some embodiments, a cavity 115 is formed on the back side of the glass chip 100 to allow a layer of SiX 120 to be coated on the back side of the glass chip 100, as described in detail in FIGS. 2A-2G. As illustrated, in some embodiments, the well 110 exposes a portion of the SiX 120 coated on the back side of the glass chip 100. While the cavity 115 is illustrated as dome shaped, it should be understood that this is for simplicity, and the cavity 115 may take any number of forms, including flat, rounded, irregular, or rectangular.

FIGS. 2A-2G illustrate an example method of making an ultrathin free-standing solid-state membrane, in accordance with the present technology. In one aspect, a method of making the ultrathin free-standing solid state membrane, the method including bonding silicon with a first side of a glass wafer, depositing a gold layer on a second side of the glass wafer, patterning the gold layer, etching the glass wafer to form a well, depositing a layer of SiX onto the second side of the glass wafer, and removing the silicon is disclosed.

In FIG. 2A, a silicon wafer 230 and a glass wafer 200 are bonded. In some embodiments, the silicon wafer 230 and the glass wafer 200 are bonded with anodic bonding. In an example, an EVG® Wafer Bonder is used for this portion of the procedure. In some embodiments, the glass wafer 200 may be cleaned before bonding. In one example, Borofloat® 33 glass wafers (500+/−25 μm thickness) were cleaned using piranha solution, then dried prior to bonding. In one example, to bond the silicon wafer 230 and the glass wafer 200, a Borofloat® 33 glass wafer 200 was positioned on top of a fresh, crystalline silicon wafer 230 (500+/−25 μm thickness), then clamped together in the EVG® bond tool. In some embodiments, the bond tool is mounted into the bonder then pumped under vacuum to a pressure of 1E-2 mTorr, after which a pump and N₂ purge cycle is performed to clear the chamber of any debris. A force, such as 500 N of force, may be applied to the top surface of the bond tool by a piston. In some embodiments, the bond tool is heated on both sides. In some embodiments, the temperature the tool is heated to is 350° C. Once at temperature, the bond tool may be pumped down to a pressure of 2.50E-3 mTorr. In some embodiments, −500V of potential is applied to the wafer stack for five minutes, during which the two wafers 200, 230 bond together. Once complete and back to room temperature, the bonded wafer 200, 230 may be removed and cleaned in piranha solution to remove any residue or debris left on the glass surface 200 from contact with the bond tool.

FIG. 2B illustrates the sputtering and patterning of a gold (or Au) layer 240 on the surface of the glass wafer 200. In some embodiments, an additional layer (not pictured) may be sputtered between the glass wafer 200 and the gold layer 240 to promote adhesion. In some embodiments, the additional layer is chromium (Cr). In some embodiments, the Cr layer is 10 nm thick. In some embodiments, the Cr layer is sputtered onto the glass wafer 200 of the bonded wafers 200, 230 at 3 nm/rotation to promote Au adhesion. In some embodiments, a gold layer 240 is then sputtered onto the glass wafer 200. In some embodiments, the Au layer 240 is 200 nm thick. In some embodiments, the Au layer 240 is sputtered onto the surface at 7 nm/rotation. After sputtering, the bonded wafers 200, 230 may be treated in an oven, such as a hexamethyldisilane (HMDS) oven to improve resist adhesion. In an example, the bonded wagers 200, 230 are spin-coated in an enclosed bowl with AZ® 10XT at 4000 rpm (5 μm film) before soft baking at 110° C. for 3 minutes. In some embodiments, a photomask is used to expose a resist layer. In one example, a custom Mylar® photomask was used to expose the resist layer at 400 mJ/cm² (400 nm). The bonded wafers 200, 230 may then be submerged in 1:4 AZ® 400K:DI Water for 4 minutes to develop. In some embodiments, the surface was washed with DI water and dried in a spin rinse dryer (SRD) tool following development. In some embodiments, the bonded wafers 200,230 may be cleaned with O₂ plasma for 5 minutes in a reactive ion etcher (RIE) to clean the surface of any excess photoresist.

In FIG. 2C, the gold layer 240 is wet etched. In some embodiments, both the Au layer 240 and the Cr layer are wet etched. With the crosslinked resist layer still intact, the bonded wafers 200, 230 may be submerged in TFA Gold Etchant for ˜30 s, then in Cr Etchant 1020 to selectively expose the back side of the upper glass layer 200. After etching, the bonded wafers 200, 230 may be washed with DI water and dried in an SRD tool.

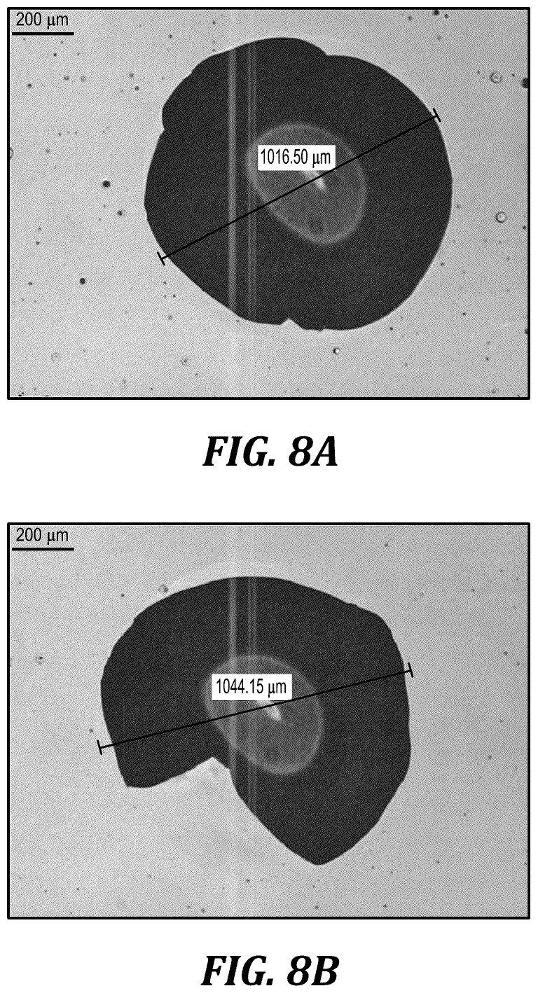

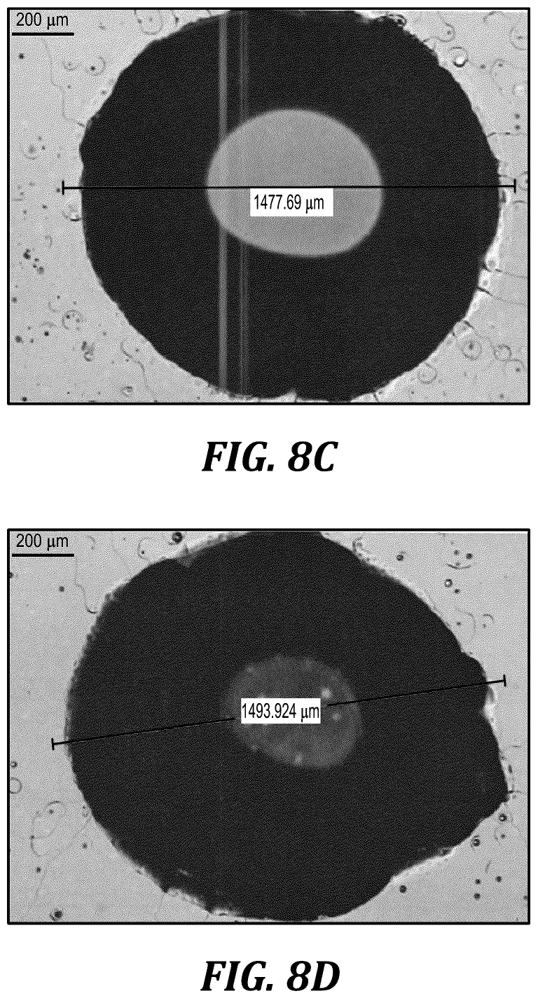

In FIG. 2D, the glass wafer 200 is hydrofluoric acid (HF) etched. In some embodiments, after drying, the bonded wafers 200, 230 are submerged in 49% HF solution for between 60 to 95 min, or until bare silicon 230 is exposed by the etched hole as shown in FIGS. 8A-8D. In some embodiments, the crosslinked resist layer is kept intact, limiting the wicking of HF through the gold layer 240. After etching, the bonded wafers 200, 230 may be washed with DI water and dried in an SRD tool. Once complete, the resist layer may be stripped with acetone or IPA. In some embodiments, the remaining Au/Cr layers 240 may be removed using the wet etching procedure described in FIG. 2C.

In FIG. 2E, a layer of SiX 220 is deposited. In some embodiments, the Six layer 220 is a layer of a silicon nitrogen compound. In some embodiments, the Six layer 220 is a silicon-oxygen compound. In some embodiments, the SiX is deposited via plasma enhanced chemical vapor deposition (PECVD). In some embodiments, the SiX is deposited via low pressure chemical vapor deposition (LPCVD). In one example, a layer of SiX (silicon nitride) 220 is deposited into the etched holes using PECVD at a rate of 0.6 nm/s. In some embodiments, the layer of SiX is patterned with dicing guidelines (such as dicing guidelines 350 in FIG. 3A). The glass wafer 200 may be diced into a plurality of glass chips, as described in further detail in FIGS. 3A-3B.

In FIG. 2F, the silicon wafer 230 is ground. In some embodiments, after SiX deposition, the silicon wafer 230 of the bonded wafers 200, 230 is ground to a desired thickness. In some embodiments, the thickness is about 70 μm. In some embodiments, the thickness ranges from about 60 to 100 μm. In some embodiments, the thickness is less than 60 μm. In some embodiments, the silicon wafer 230 is ground using a wafer back grinder. After grinding, the glass wafer 200 may be diced into a plurality of glass chips (as described in FIG. 3B).

In FIG. 2G, the silicon wafer 230 is removed from the glass wafer 200, to form the membrane 2000. In some embodiments, the diced glass chips are mounted onto a second silicon wafer with the silicon surface facing out (not pictured in FIG. 2G). In some embodiments, the chips are mounted using Santovac® 55 vacuum grease. In some embodiments, the second silicon wafer is etched using deep reactive ion etching (DRIE) to thin the second silicon wafer. In some embodiments, the second silicon wafer is thinned to a thickness of about 1 μm. This thinning may be indicated by a shift in color from dark grey to a deep red. In some embodiments, the glass chips are placed in a KOH solution to remove any remaining silicon. In one example, thinned chips were placed in 2M KOH solution held at 60° C. to remove any remaining silicon. The final devices may be washed with DI water then dried with N₂ gas. A membrane 2000 may be formed, having a layer of SiX 220, and an etched glass wafer (or chip) 200, forming a well 210. In some embodiments, one or more nanopores may be formed on the Six. In some embodiments, a single nanopore may be formed in the SiX so that the nanopore is exposed by the well. In some embodiments, a plurality of nanopores may be formed in the Six so that the well exposes the plurality of nanopores. In some embodiments, such as that illustrated in FIG. 1A, each well on a glass wafer or chip may expose one or a plurality of nanopores.

FIG. 3A is an example glass wafer 300 having dicing markers 350, in accordance with the present technology. As described in FIG. 2E, the glass wafer 300 may be diced into a plurality of glass chips (as shown in FIG. 3B). In some embodiments, a photomask is used to pattern dicing guidelines 350 onto the SiX (and thus the glass wafer 300). While the dicing guidelines 350 are patterning squares in FIG. 3A, the dicing guidelines 350 can pattern the glass wafer 300 in any number of shapes. In an example, 11.1×11.1 mm squares were patterned onto the nitride surface (or SiX layer 220) as dicing guidelines 650. In some embodiments, the photomask is a custom Mylar® photomask. In some embodiments, the same resist/exposure procedure as that is used to pattern the gold layer 240 in FIG. 2C is used to pattern the SiX.

FIG. 3B is a plurality of glass chips 360A, 360B, 360C formed from the glass wafer 300 along the dicing markers 350 of FIG. 3A, in accordance with the present technology. In some embodiments, after grinding the silicon wafer 230, the glass wafer 300 is diced into chips along the patterned dicing lanes (markers) 350. In some embodiments, the glass wafer 300 is diced using a DISCO® automatic dicing saw with a 52 mm diameter flanged diamond blade. After dicing, the glass chips 360A, 360B, 360C may be separated, as shown in FIG. 3B. In some embodiments, the remaining resist is stripped with acetone/IPA.

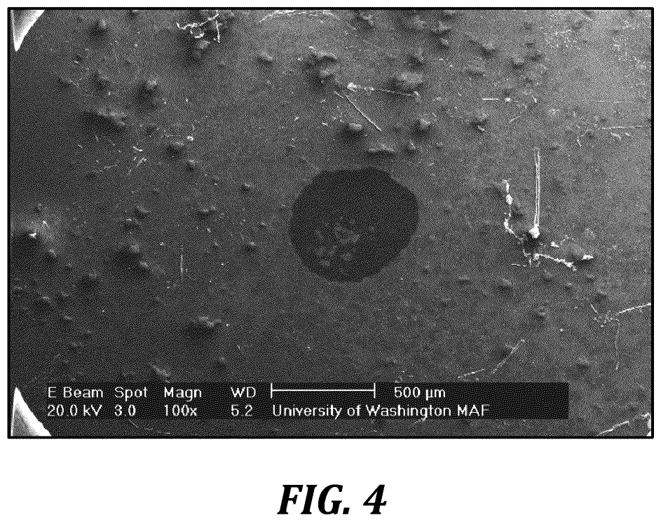

FIG. 4 is an SEM image of an example well of an ultrathin free-standing solid-state membrane, such as membrane 1000, in accordance with the present technology. As indicated on the bar on the bottom of FIG. 4, the SEM image was taken with electron beam (or e-beam) at 20.0 kV. The magnification is 100×, the focal length (or working distance (WD)) is 5.2, and the scale bar represents 500 μm. A well (such as well 110), is shown as a dark, round shape in the center of the image, while the glass chip (such as glass chip 100) is lighter grey and surrounds the well. As shown in the SEM image, the well reveals the layer of Six patterned on the backside of the silicon wafer. In some embodiments, the well is about 500 μm in diameter.

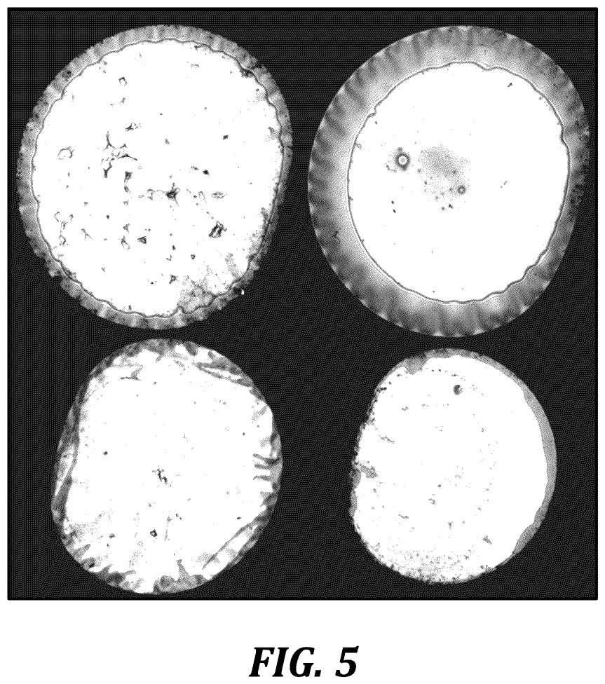

FIG. 5 is a composite of four brightfield micrographs of four different wells located on a single glass wafer, in accordance with the present technology. In some embodiments, such as that shown in FIG. 1A, the glass chip includes a plurality of wells. FIG. 5 is not to scale, but four wells on the same glass chip are shown. The dark background is the glass chip, while the bright circular shapes are wells, exposing white SiX patterned on the backside of the glass chip. The SiX can also be seen under the thinned glass, represented by the light grey forms surrounding the four wells.

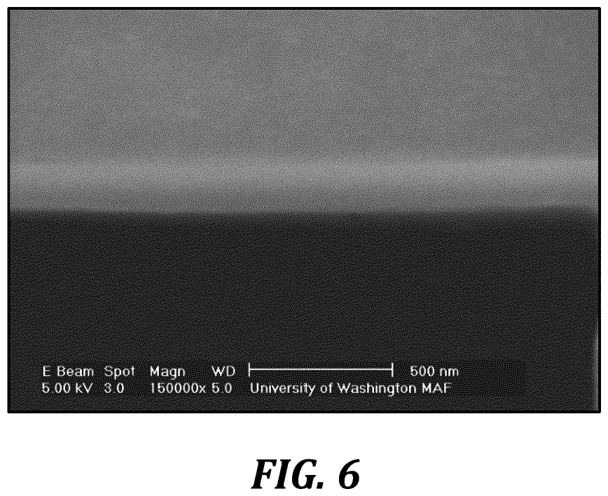

FIG. 6 is an SEM image of the thickness of an example ultrathin free-standing solid-state membrane, in accordance with the present technology. As shown in the bar labeling FIG. 6, the SEM image was taken with an e-beam of 5.00 kV at a magnification of 150000× and a focal length (WD) of 5.0. The scale bar represents 500 nm. As shown, the thickness of the silicon nitride may be about 300 nm. In some embodiments, the thickness of the silicon nitride ranges from 250 nm-1000 nm.

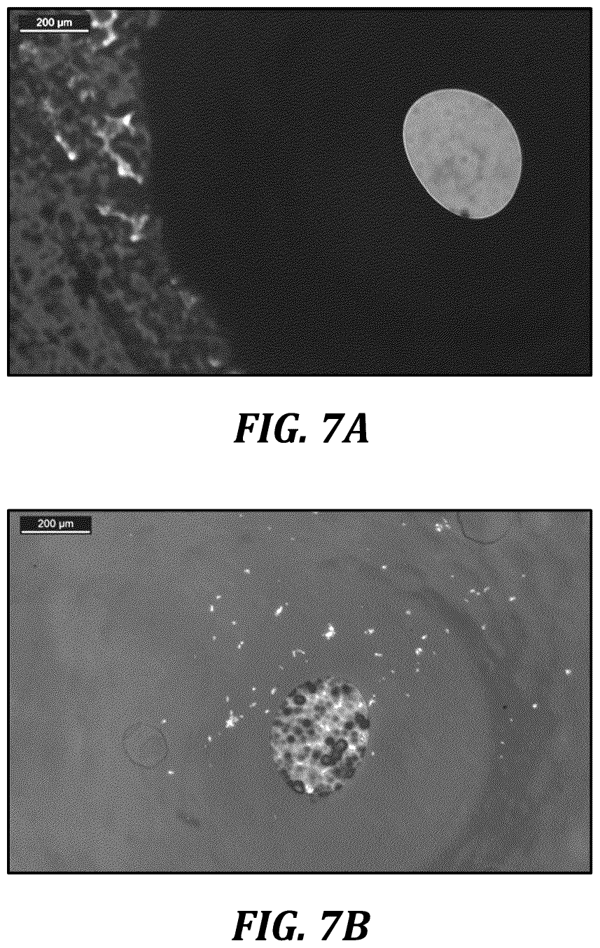

FIG. 7A is a brightfield micrograph of a silicon oxide ultrathin free-standing solid-state membrane, in accordance with the present technology. In some embodiments, the layer of Six may be a layer of silicon oxide. In the top left corner of the image is a scale bar representing 200 μm. A well is shown, represented by the light grey oval in the right-hand side of the micrograph. Glass surrounds the well, as shown by the black region of the image. A thin line of lighter grey surrounding well shows that the silicon oxide is visible in the thinned regions of the glass.

FIG. 7B is a brightfield micrograph of another silicon oxide ultrathin free-standing solid-state membrane, in accordance with the present technology. In the top left corner of the image is a scale bar representing 200 μm. The well is shown in the bottom center of the image. As in FIG. 7A, the silicon oxide is exposed by the well.

FIG. 8A-8D are SEM images of example HF etched glass chips, in accordance with the present technology. In the top left corner of the image is a scale bar representing 200 μm. A well is shown in each of FIGS. 8A-8D, represented as a grey circular shape in the center of the image. The lighter background is glass. The glass is etched until the bonded silicon wafer is exposed by the well, as described in FIG. 2D. FIGS. 8A-8D show variability between wells in the HF etching process as described in FIG. 2D. In some embodiments, the glass wafer is etched for 60 to 95 minutes.

FIGS. 8A and 8B were etched for 60 minutes. The 1-hour HF etch in FIG. 8A resulted in a well having a diameter of 1016.50 μm on the backside of the glass wafer. In some embodiments, the well is not circular, but may take any form, such as an organic or irregular shape. In FIG. 8B, the 1-hour HF etch resulted in an irregular well with a diameter of 1044.15 μm at the backside of the glass wafer.

FIG. 8C shows a 95-minute HF etch that resulted in a well with a diameter of 1477.69 μm. Finally, FIG. 8D shows a 90-minute HF etch that resulted in a well with a diameter of 1493.924 μm. In some embodiments, the etched well ranges from about 200 μm to about 5500 μm in diameter at the backside of the glass wafer.

The membrane as described herein may be used for any number of applications, including for MEMS device scaffolding, DNA sequencing, TEM imaging, microparticle analysis, nanoparticle analysis, medicinal applications, environmental applications, electrochemical applications, or mechanical applications.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

It will be appreciated that, although specific embodiments of the invention have been described herein for purposes of illustration, various modifications may be made without deviating from the spirit and scope of the invention. Accordingly, the invention is not limited except as by the claims.

While illustrative embodiments have been illustrated and described, it will be appreciated that various changes can be made therein without departing from the spirit and scope of the invention.

1. An ultrathin free-standing solid state membrane, comprising:
an etched well on a glass wafer;
a cavity disposed on a backside of the glass wafer; and
a layer of SiX deposited into the cavity.

2. The membrane of claim 1, wherein the layer of SiX is between 10 nm and 2000 nm thick.

3. The membrane of claim 1, wherein the glass wafer is selected from quartz glass, borosilicate glass, or a combination thereof.

4. The membrane of claim 1, wherein the SiX is a silicon-nitrogen compound.

5. The membrane of claim 1, wherein the SiX is a silicon-oxygen compound.

6. The membrane of claim 1, wherein the etched well ranges from about from about 200 μm to about 5500 μm in diameter.

7. The membrane of claim 1, wherein the etched well comprises a plurality of nanopores.

8. The membrane of claim 1, wherein the etched well is one of a plurality of etched wells on the glass wafer.

9. The membrane of claim 8, wherein the etched wells of the plurality etched of wells are arranged in an array.

10. A method of making the ultrathin free-standing solid state membrane of claim 1, the method comprising:
bonding silicon with a first side of a glass wafer;
depositing a gold layer on a second side of the glass wafer;
patterning the gold layer;
etching the glass wafer to form a well;
depositing a layer of SiX onto the second side of the glass wafer; and
removing the silicon.

11. The method of claim 10, wherein the SiX is deposited via plasma enhanced chemical vapor deposition (PECVD).

12. The method of claim 10, wherein the SiX is deposited via low pressure chemical vapor deposition (LPCVD).

13. The method of claim 10, wherein the SiX is a silicon-nitrogen compound.

14. The method of claim 10, wherein the SiX is a silicon-oxygen compound.

15. The method of claim 10, wherein the method further comprises:
patterning the SiX to form dicing guidelines; and
dicing the glass wafer into glass chips along the dicing guidelines.

16. The method of claim 15, wherein the method further comprises placing the glass chips in a KOH solution to remove any remaining silicon.

17. The method of claim 10, wherein removing the silicon comprises etching the silicon with a deep reactive ion etch (DRIE).

18. The method of claim 10, wherein the method further comprises forming a plurality of nanopores on the SIX.

19. The method of claim 10, wherein the method further comprises forming a plurality of wells on a single glass wafer.

20. A method of using the membrane of claim 1 for MEMS device scaffolding, DNA sequencing, TEM imaging, microparticle analysis, nanoparticle analysis, medicinal applications, environmental applications, electrochemical applications, or mechanical applications.
